(19) United States
(12) Patent Application Publication
(10) Pub. No.: US 2025/0000436 A1
(43) Pub. Date: Jan. 2, 2025

(54) AI-Based Tool for Assessing Motor Visuo-Spatial Gestalt and Memory Abilities

(71) Applicant: Bar-Ilan University, Ramat Gan (IL)

(72) Inventors: Rachel Schiff, Givat Shmuel (IL); Roi Yozevitch, Nofim (IL)

(73) Assignee: Bar-Ilan University, Ramat Gan (IL)

(21) Appl. No.: 18/291,980

(22) PCT Filed: Jul. 25, 2022
(86) PCT No.: PCT/IL2022/050801

§ 371 Date: Jan. 25, 2024

Related U.S. Application Data
(60) Provisional application No. 63/226,776, filed on Jul. 29, 2021.

(51) Int. Cl. A61B 5/00; A61B 5/16; G06F 3/0354

(52) U.S. Cl. CPC A61B 5/4088; A61B 5/162; A61B 5/7267; A61B 5/7475; G06F 3/03545

(57) ABSTRACT

A method of evaluating a subject's visuospatial ability includes: displaying an original drawing on a screen; receiving input on the screen from a stylus, corresponding to a reproduction of the drawing by the subject; during the receiving step, collecting metadata regarding the process of the reproduction; comparing a resemblance of the reproduction to the original drawing; evaluating the reproduction and the metadata to infer therefrom the subject's understanding of the drawing; and issuing a combined evaluation of the subject's gestalt understanding of the drawing based on a combination of the resemblance comparison and the metadata evaluation.

### RELATED APPLICATIONS

This application is the United States National Stage of International Patent Cooperation Treaty Patent Application No. PCT/IL2022/050801, filed Jul. 25, 2022, which claims the benefit of U.S. Provisional Patent Application No. 63/226,776, filed Jul. 29, 2021, each hereby incorporated by reference herein.

### FIELD OF THE INVENTION

The present disclosure, in some embodiments, concerns a system and method for assessing motor visuo-spatial gestalt and memory abilities. More specifically, but not exclusively, the disclosure is directed to a system for implementing a traditional pen-and-paper gestalt test on a digital platform, for recording metadata regarding a subject's reproduction of an image, and for combining analysis of that metadata with analysis of the reproduction in order to issue a combined evaluation of the subject's gestalt and memory abilities.

### BACKGROUND OF THE INVENTION

Various tests involving production or reproduction of drawings are currently in widespread use, for evaluation of motor or neuropsychological impairments. One commonly used test is the Bender Visual-Motor Gestalt Test (hereinafter, the Bender test). The Bender test is a psychological test used by mental health practitioners that assesses visual-motor functioning, developmental disorders, and neurological impairments in children ages 3 and older and adults. The test consists of a series of index cards picturing different geometric designs. The cards are presented individually and test subjects are asked to copy the design before the next card is shown. Test results are scored based on the accuracy and organization of the reproductions. The Bender test has been in use, in various versions, since 1938.

Another test that requires subjects to copy patterns from cards is the Beery-Buktenica Developmental Test of Visual-Motor Integration. The subject is presented a sequence of images and is asked to copy the images, beginning from a simple line and progressing gradually to more complex geometric shapes. The test assesses how the visual perceptual and fine motor control systems coordinate with one another, or, in other words, how well the motor system produces what the visual system is processing.

Other tests involving production or reproduction of drawings include: the Rey-Osterrieth Complex Figure Test for evaluation of visual perception and long term visual memory; the Clock Draw Test, which is a screening test for people with cognitive impairments and dementia; the Osborn, Butler, & Morris (1984) Copy Design task, which instructs children to copy eight simple geometric designs; and the Trail-Making-Test, which calls upon the subject to connect between a set of numbered dots, in numerical order, without lifting the pen from the page.

Each of the tests described above has traditionally been performed with pen and paper. An exemplary implementation of a drawing reproduction test is shown in FIG. 1A. On the left, a card 1 is shown to the subject with a pattern 2 printed thereon. The subject reproduces the pattern on a sheet of paper 3, by using writing implement 4 (e.g., a pencil or pen) to draw reproduced pattern 5. The practitioner collects each paper 3 with a reproduced pattern 5, and manually grades the quality of the reproductions. The grading considers, inter alia, the presence or absence of certain properties such as rotation, retrogression, angulation, simplification, and closure difficulty.

In recent years, image processing technology has been implemented to complement human scoring of pen-and-paper psychological tests. In one example, pen-and-paper examples of the Bender Gestalt test were scanned and uploaded to a computer for processing. The processing included techniques such as segmentation, counting number of drawing components, and computing an area of a bounding box surrounding the drawing. The computer program was programmed to evaluate the drawings on the basis of factors such as simplification, overlapping difficulty, rotation, and perseveration.

In addition, certain psychological tests that have previously been performed off-line have been transitioned to digital platforms. For example, the Corsi block-tapping test requires a subject to mimic a researcher as he or she taps a sequence of up to nine identical spatially separated blocks. The Corsi block-tapping test has been implemented successfully on a tablet computer for several years. More recently, an adaptation of the Trail-Making Test has also been implemented on a tablet.

### SUMMARY OF THE INVENTION

Pen-and-paper gestalt tests, even when analyzed by a computer, are unable to capture all relevant information regarding the subject's understanding. For example, image processing is unable to evaluate factors such as time spent, pen pressure, or speed. In addition, existing digitized psychological tests, such as the Corsi or Trail-Making Tests, measure only a limited number of parameters, in particular time spent and correctness of input. Many factors relevant to gestalt understanding and memory, such as pen pressure, pen angle, and number of pen lifts, are not relevant to those implementations.

Accordingly, there is a need for a computer-based gestalt test that is able to collect metadata relevant to a patient's visuo-spatial gestalt and memory abilities, and to integrate this metadata into a comprehensive scoring system including evaluation of the patient's reproduction and the metadata. There is also a need for a computer-based gestalt test that is capable of not only of identifying the content of what is drawn by the subject, but also of ranking the quality of the reproduction.

The present disclosure discloses a tablet-based implementation of a pattern-copying test. The tablet displays a drawing on a screen, and prompts the user to reproduce the drawing. A processor collects metadata regarding the user's execution of a copying of the drawing. After the user completes the copy, the processor evaluates the quality of the reproducing using image processing techniques. The metadata and image processing evaluation are fed into separate neural networks. The neural networks output a combined quality score of the reproduction, derived both from the image processing and from the metadata. The processor further outputs the collected metadata as vectors, for separate analysis.

According to a first aspect, a method of evaluating a subject's visuospatial ability is disclosed. The method includes: displaying an original drawing on a screen; receiving input on the screen from a stylus, corresponding to a reproduction of the drawing by the subject; during the receiving step, collecting metadata regarding the process of the reproduction; comparing a resemblance of the reproduction to the original drawing; evaluating the reproduction and the metadata to infer therefrom the subject's understanding of the drawing; and issuing a combined evaluation of the subject's gestalt understanding of the drawing based on a combination of the resemblance comparison and the metadata evaluation.

In another implementation according to the first aspect, the metadata include a reaction time from displaying of the original drawing until commencement of reproduction of the drawing, and a performance time from commencement of reproduction until completion of reproduction.

In another implementation according to the first aspect, the metadata include a number of stylus strokes used to reproduce the drawing.

In another implementation according to the first aspect, the metadata include pressure exerted by the subject with the stylus onto the screen during reproduction.

In another implementation according to the first aspect, the metadata include azimuth angle of the stylus on the screen during reproduction.

In another implementation according to the first aspect, the metadata include age of the subject.

In another implementation according to the first aspect, the comparison of resemblance is based on at least: position of reproduction on the screen, scale of reproduction, orientation of reproduction, and number of elements in the reproduction.

In another implementation according to the first aspect, the method further includes repeating the method with a series of predefined original drawings. Optionally, the metadata include cumulative time of completion of all the predefined original drawings in the series.

In another implementation according to the first aspect, the method further includes: performing the method with a plurality of unique subjects; aggregating the metadata collected from each subject; and, based on the aggregated metadata, determining a kernel density function for performance in one or more measured metadata categories, and deriving a norm for standard performance from the kernel density function.

In another implementation according to the first aspect, the method further includes: comparing the resemblance with a convolutional neural network; evaluating the metadata with a feed-forward neural network; and combining the convolutional neural network and the feed forward neural network at a dense layer phase.

Optionally, the method further includes training the convolutional neural network, the training step comprising: collecting a data set comprising a plurality of sample reproductions of the original drawings; and manually assigning a similarity score to each reproduction.

Optionally, the method further includes augmenting the data set with modified sample reproductions, said augmenting comprising at least one of changing scaling, shifting pixels, and horizontal flipping, in a manner that is sufficiently subtle not to affect a scoring of a given reproduction.

In another implementation according to the first aspect, the combined evaluation is a ranking of the subject's gestalt understanding of the original drawing.

According to a second aspect, a system for evaluating a subject's visuospatial ability is disclosed. The system includes: a mobile computing device including a touch screen configured to display images and to receive input from a stylus; a processor, and a non-transitory computer readable medium; and a computer program product embodied on the non-transitory computer readable medium. When executed by the processor, the computer program product causes the processor to perform the following steps: displaying an original drawing on the screen; during receipt of input on the screen from the stylus, corresponding to a reproduction of the drawing by the subject; collecting metadata regarding the process of the reproduction; comparing a resemblance of the reproduction to the original drawing; evaluating the reproduction and the metadata to infer therefrom the subject's understanding of the drawing; and issuing a combined evaluation of the subject's gestalt understanding of the drawing based on a combination of the resemblance comparison and the metadata evaluation.

In another implementation according to the second aspect, the metadata include a reaction time from displaying of the original drawing until commencement of reproduction of the drawing, and a performance time from commencement of reproduction until completion of reproduction.

In another implementation according to the second aspect, the metadata include a number of stylus strokes used to reproduce the drawing.

In another implementation according to the second aspect, the metadata include pressure exerted by the subject with the stylus onto the screen during reproduction.

In another implementation according to the second aspect, the metadata include azimuth angle of the stylus on the screen during reproduction.

In another implementation according to the second aspect, the metadata include age of the subject.

In another implementation according to the second aspect, the computer program product is configured to compare the resemblance based on at least: position of reproduction on the screen, scale of reproduction, orientation of reproduction, and number of elements in the reproduction.

In another implementation according to the second aspect, the computer program product is configured to repeat each of the steps with a predefined series of original drawings. Optionally, the metadata include cumulative time of completion of all the predefined original drawings in the series.

In another implementation according to the second aspect, the computer program product is further configured to aggregate metadata collected from a plurality of unique subjects, and, based on the aggregated metadata, determine a kernel density function for performance in one or more measured metadata categories, and derive a norm for standard performance from the kernel density function.

In another implementation according to the second aspect, the computer program product further comprises a convolutional neural network for comparing the resemblance, and a feed forward neural network for evaluating the metadata, as wherein the convolutional neural network and the feed forward neural network at a dense layer phase, so as to output a single combined evaluation.

In another implementation according to the second aspect, the combined evaluation is a ranking of the subject's gestalt understanding of the original drawing.

### BRIEF DESCRIPTION OF THE DRAWINGS

In the drawings:

FIG. 1A depicts a prior art pen-and-paper implementation of a gestalt test;

FIG. 1B depicts a tablet-based implementation of a gestalt test, according to embodiments of the present disclosure;

FIGS. 2A-2D depict sample original drawings to be used in the gestalt test, according to embodiments of the present disclosure;

FIGS. 3A-3C depict original drawings and reproductions, illustrating factors relevant to the quality of the reproduction, according to embodiments of the present disclosure;

FIGS. 3D-3F depict original drawings and reproductions, illustrating factors relevant to the process of reproduction and measured by metadata, according to embodiments of the present disclosure;

FIG. 4 depicts steps of a method of testing a subject's gestalt understanding, according to embodiments of the present disclosure;

FIG. 5 schematically depicts a structure of a combined neural network used to produce a combined output for an evaluation of a subject's reproduction, according to embodiments of the present disclosure;

FIGS. 6A-6E depict an original drawing and four reproductions of the original drawing that were assigned different scores, according to embodiments of the present disclosure;

FIGS. 7A-7B depict examples of kernel density estimation of the time taken to complete a drawing for two groups of subjects, according to embodiments of the present disclosure; and

FIGS. 8A-8B illustrate generation of norms from the data generated from multiple groups of subjects, according to embodiments of the present disclosure.

### DETAILED DESCRIPTION OF THE INVENTION

The present disclosure, in some embodiments, concerns a system and method for assessing motor visuo-spatial gestalt and memory abilities. More specifically, but not exclusively, the disclosure is directed to a system for implementing a traditional pen-and-paper gestalt test on a digital platform, for recording metadata regarding a subject's reproduction of an image, and for combining analysis of that metadata with analysis of the reproduction in order to issue a combined evaluation of the subject's gestalt and memory abilities.

Before explaining at least one embodiment in detail, it is to be understood that the invention is not necessarily limited in its application to the details of construction and the arrangement of components and/or methods set forth in the following description and/or illustrated in the drawings. The invention is capable of other embodiments or of being practiced or carried out in various ways.

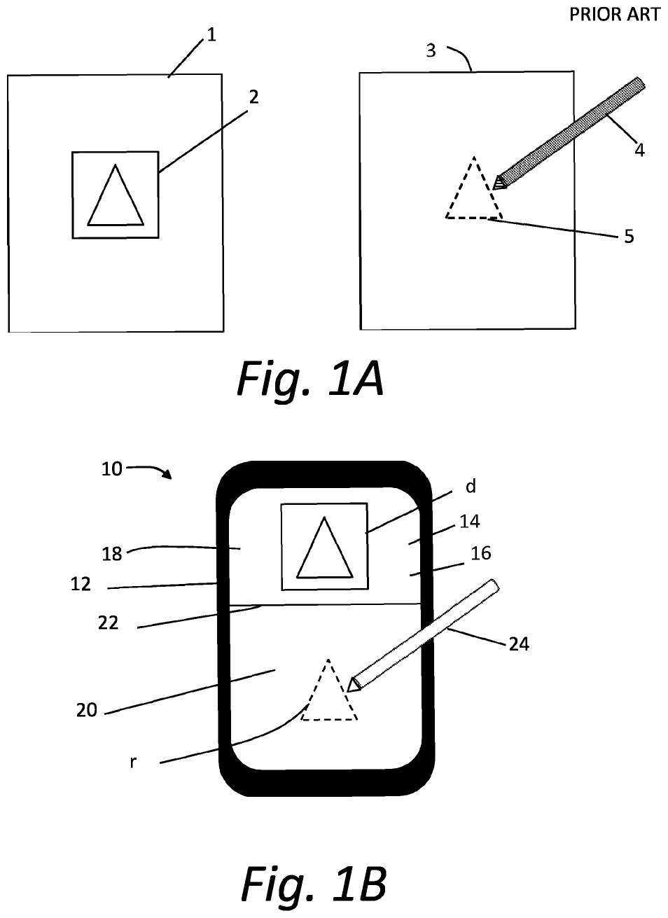

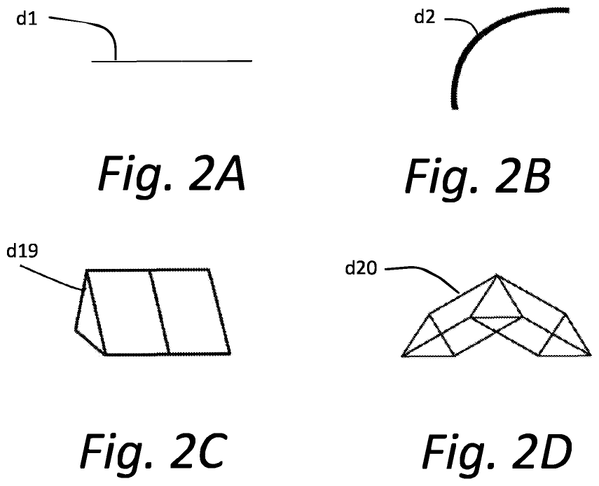

FIG. 1B depicts components of a system 10 for evaluating a subject's visuospatial gestalt and memory ability. The system includes mobile computing device 12. In exemplary embodiments, mobile computing device 12 is a tablet computer. Henceforth, the terms “mobile computing device” and “tablet” will be used interchangeably.

Tablet 12 includes a touch screen 14. The touch screen 14 may be any standard screen or display suitable for implementation in a mobile computing device, such as LCD, OLED, AMOLED, Super AMOLED, TFT, or IPS.

Touch screen 14 is configured to display a graphic user interface 16. The graphic user interface 16 includes a first region 18, in which an original drawing d is displayed, and a second region 20, which includes an open space for a user to reproduce the original drawing d with reproduction r. Optionally, one or more demarcating lines 22 are also displayed, in order to form a boundary between the display region 18 and the reproduction region 20. In the illustrated embodiment, the display region 18 is depicted as being above the reproduction region 20; this configuration is merely exemplary, and other configurations are also possible.

In an alternative testing scenario, the subject is required to retrieve an original drawing from memory following being shown that drawing, and to draw the drawing solely based on memory. In such a scenario, the original drawing may be displayed in the display region 18 for a given period of time, and then erased from the display region 18. Alternatively, the original drawing may be displayed on the entire display 14, and then removed from the display 14.

System 10 also includes stylus 24. Stylus 24 is an active stylus, also known as a digital stylus. An active stylus has digital components or circuitry inside the stylus 24 that communicate with a digitizer of the tablet 12. This communication allows for measurement of features such as pressure sensitivity, tilt, number of pen raises, and timing. Examples of active styluses currently available include the Apple Pencil® and the Microsoft Surface Pen®. The Surface Pen, for example, is capable of detecting 4,096 levels of pressure and has 1,024 levels of tilt sensitivity. In experiments implementing the system and method disclosed herein, the tablet that was used was the Microsoft Surface Pro® 7, and the stylus was the Surface Pen®, version 2.

Tablet 12 also includes a processor (not shown). The processor includes a memory, and circuitry for executing computer readable program instructions stored on the memory. The memory is a non-transitory storage medium having stored thereon code instructions that, when executed by the processor, causes performance of various steps. The storage medium may be, for example, an electronic storage device, a magnetic storage device, an optical storage device, a semiconductor storage device, or any suitable combination of the foregoing. In particular, the functions described herein may be programmed a computer program product installed on the non-transitory computer readable medium of tablet 12. In addition, the functions described herein may be performed by a cloud-based computer, or by a combination of a processor and memory stored on the tablet and a processor and memory stored on a remote device.

Mobile device 12 is further equipped with a communications module for wirelessly communicating with the cloud, for example, via a Bluetooth or wireless internet connection. This wireless connection is used, inter alia, for downloading software updates, including updates to the deep neural networks disclosed herein.

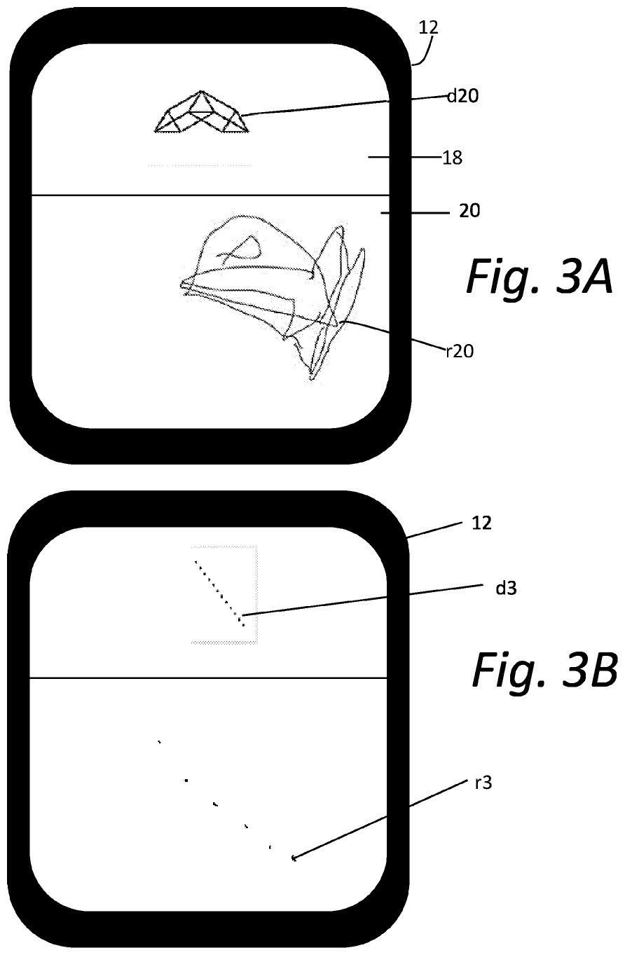

FIGS. 2A-2D depict different geometrical shapes that may be presented to a subject for reproduction. In exemplary embodiments, the subject is a child. During an exemplary testing session, the subject is presented with a series of twenty original drawings that he or she is required to copy, while viewing the original drawings, and twenty original drawings that he or she is required to retrieve, following removal of the original drawings from display. The shapes used for the copying and retrieval tasks are of a similar level of difficulty to each other. In addition, the drawings present the subject with a progressive challenge due to their increasing complexity. For example, the first two drawings in a series are drawing d1, which is a simple line, and drawing d2, which is a simple curve. The final two drawings in a series are d19 and d20, which involve complex geometric shapes formed of multiple intersecting lines.

As discussed above, each reproduction is evaluated based on two sets of criteria: the objective quality of the reproduction, as measured by the elements of the reproduction that the subject produced; and metadata collected during the process of the reproduction.

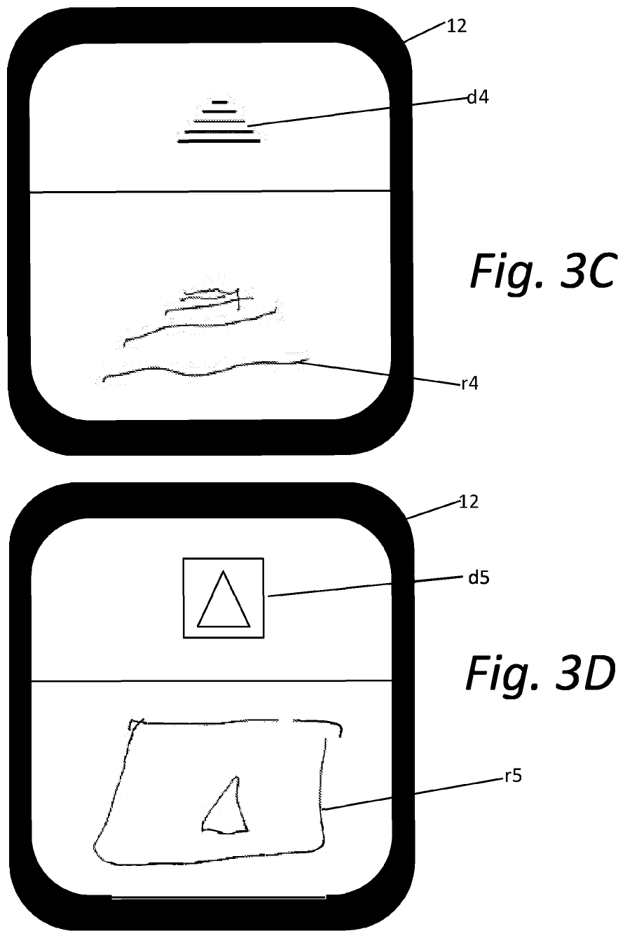

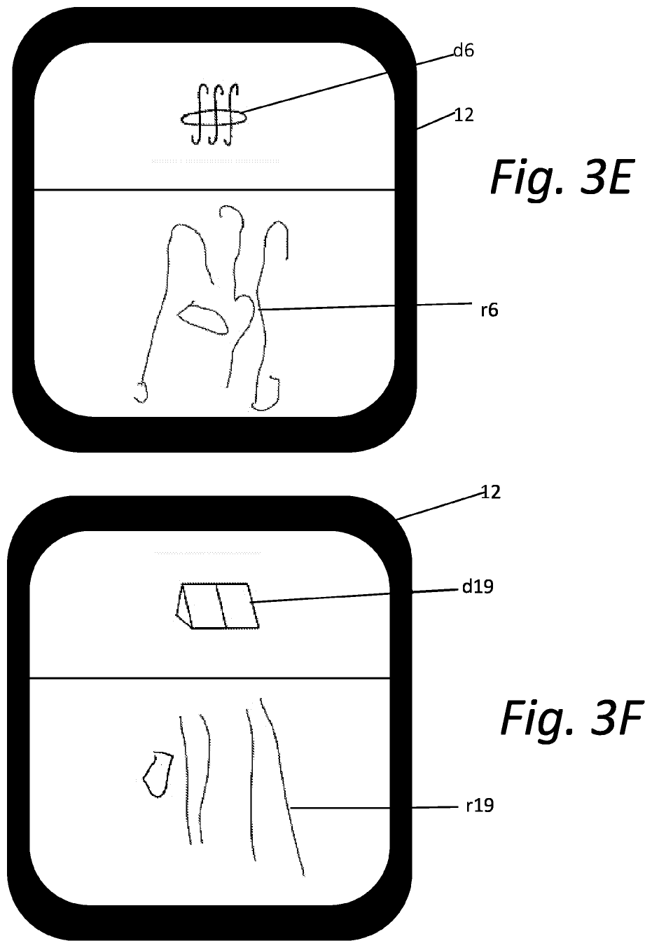

FIGS. 3A-3C depict different examples of reproductions of original drawings. Each reproduction highlights a different feature or features that may be evaluated based solely on the quality of the reproduction.
Position, Scale, and Orientation. Referring to FIG. 3A, original drawing d20 is positioned in the center of the display region of the tablet 12. Reproduction r20 is displaced to the right side of the reproduction region 20, and is also significantly larger than the original drawing d20. In addition, there are lines slanted toward the upper right corner of the reproduction region 20, but there are no lines slanted toward the upper left corner, as in the original drawing d20. Thus, this subject displayed a poor understanding of original drawing d20.
Number of Elements. Referring to FIG. 3B, original drawing d3 consists of twelve dots arranged in a diagonal line. However, duplication r3 consists of only six dots. Thus, the subject exhibited poor gestalt understanding of the original drawing. By contrast, in FIG. 3C, original drawing d4 shows five horizontal lines. Reproduction r4 also contains five semi-horizontal lines. Thus, the subject correctly understood that the drawing contained five lines. Although the orientation and size are slightly displaced compared to the original drawing d4, overall this subject exhibited good gestalt understanding of the original drawing d4. Another metric that is relevant to FIG. 3C is “Horizontal Line Ratio.” The horizontal line ratio is the ratio between the length and width of a horizontal line. A perfectly horizontal line would have a width of zero; however, virtually all reproductions of horizontal lines in these contexts, including those of FIG. 3C, have some width. The horizontal line ratio thus illustrates the degree to which the line was drawn in a straight and precise manner.

The above-mentioned features may be evaluated without regard to metadata concerning how the reproductions were generated. This evaluation may be performed with both standard computer vision methods and with artificial neural networks. In exemplary embodiments, a computer vision algorithm initially extracts the reproduction's position, as determined by the center of mass, and further analyzes the image's scale, the orientation, the centering, and the skew. On the basis of such evaluations, the system is able to not only determine the content of what is drawn (for example, that there are five lines) but also provide an evaluation of the quality of the reproduction.

As discussed above, system 10 is also capable of collecting metadata regarding the process of formation of the reproduction. As used in the present disclosure, the term metadata encompasses all relevant information about how an image is generated. The metadata includes, but is not limited, to data collected through sensors in stylus 24. For example, in addition to the examples described below, the metadata may encompass the age of the subject, the location of the subject, and the time during which the examination was performed.

The following metadata are collected over the course of each reproduction, before, during, and after receipt of input on the screen 14 from the stylus 24:
Reaction Time. Reaction time refers to the time elapsed from the displaying of the original drawing on display 14 of the tablet 12 until the commencement of reproduction, i.e., until the stylus 24 first touches the surface of display 14.
Performance Time. Performance time refers to the time elapsed from the commencement of reproduction, i.e., from the first pen stroke during a reproduction, to completion of reproduction with the last pen stroke. Performance time may also be referred to as “action time.” The reaction time and performance time may be measured on the nanosecond scale.
Cumulative Time. Cumulative Time refers to the total time required by the subject to reproduce all of the drawings.
Pressure. The pressure that the subject exerts with the stylus 24 onto the screen 14 is recorded continuously during the reproduction process.
Azimuth. The system 10 continuously records the azimuth angle of the stylus 24 on the screen 14 during reproduction.
Number of Pen Lifts and Strokes. An active stylus is able to measure the number of pen lifts that a user performs during a single drawing session. The number of strokes used to complete a drawing is the same as the number of pen lifts, plus one. Each stroke may be referred to herein a “pen stroke” or a “stylus stroke.” The number of strokes is considered important in psychological assessment since it is indicative of the child's gestalt understanding of the image. FIG. 3D depicts drawing d5, which is a triangle within a square. A subject that understands the drawing would understand consists of two separate shapes, one inside the other, and would draw a reproduction using two strokes—one for the triangle, and a second for the square. In the illustrated example, reproduction r5 indeed is drawn using two strokes, showing a good gestalt understanding of the drawing. By contrast, the subject in FIG. 3E was presented with a drawing d6 having four elements—the oval and the three elongated curves crossing the oval. In the example of FIG. 3E, the subject completed the reproduction r6 with six strokes instead of four. This indicates a poor Gestalt understanding.

It should be understood, of course, that number of pen strokes is not the sole criterion by which gestalt comprehension should be evaluated. There may be examples in which the number of pen strokes is as expected, but the subject exhibited a poor understanding of the drawing. Such is the case in FIG. 3F, in which the subject executed the drawing in five pen strokes, which is consistent with what one who understood the drawing might do (for example, the triangle on the left as one stroke, and each of the other four lines as its own stroke). However, even a simple visual examination of the drawing suffices to demonstrate that the subject did not understand the drawing at all-none of the lines intersect or cross as they should.

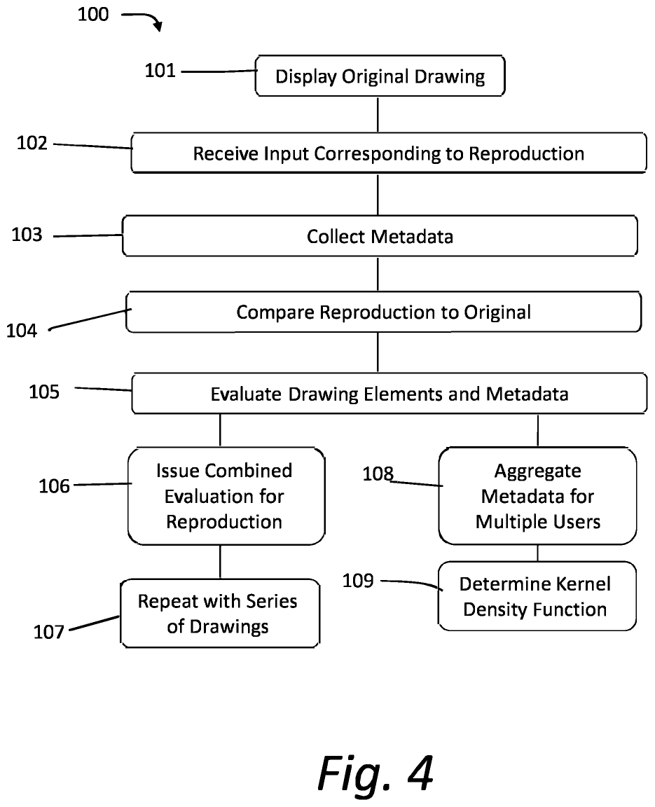

FIG. 4 illustrates steps of a method 100 of evaluating a subject's visuospatial ability, using system 10, according to embodiments of the present disclosure.

At step 101, the practitioner displays an original drawing on display 14 of the tablet 12. For example, the practitioner may execute a computer program stored on the memory of the tablet 12.

At step 102, the tablet 12 receives input from the subject corresponding to a reproduction of the original drawing. The input consists of one or more pen strokes of the stylus 24, as described above.

At step 103, during the reproduction of the original drawing, the tablet 12 collects metadata regarding the process of the reproduction. As discussed above, the metadata may include a reaction time from displaying of the original drawing until commencement of reproduction of the drawing; a performance time from commencement of reproduction until completion of reproduction; the number of stylus strokes used to reproduce the drawing; pressure exerted by the subject onto the screen during reproduction; and azimuth angle of the stylus on the screen during reproduction. Metadata such as age may be input into the tablet 12 by the practitioner.

At step 104, the system compares the reproduction to the original drawing. As discussed above, this comparison may be based on one or more of the following factors: position of reproduction on screen, scale of reproduction, orientation of reproduction, and number of elements in the reproduction.

At step 105, the system evaluates elements of the drawing and the collected metadata in order to infer therefrom the subject's gestalt understanding of the drawing. For example, as discussed above, the number of drawing elements may be compared to an expected number of drawing elements, the number of pen lifts may be compared to a number of expected pen lifts, and a time of completion may be compared to an expected time of completion. Step 105 may be performed before, during, or after step 104.

At step 106, the system issues a combined evaluation of the subject's gestalt understanding of the drawing, based on a combination of the resemblance comparison and the metadata evaluation. The combined evaluation may be a ranking of the subject's gestalt understanding, on a linear scale.

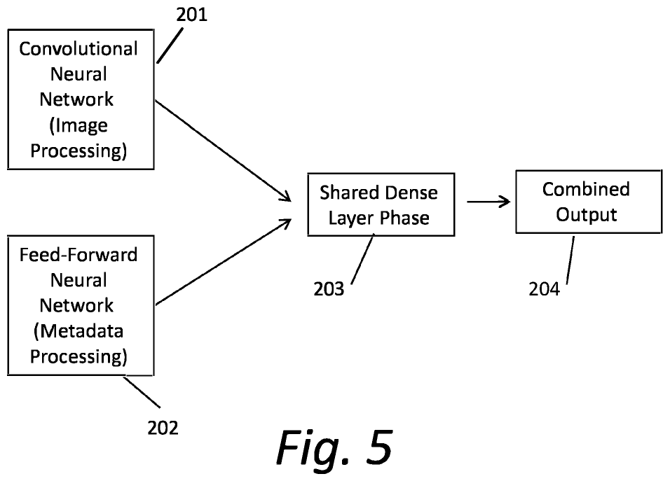

FIG. 5 schematically illustrates a structure of a series of neural networks that may be employed to perform steps 104-106.

Convolutional neural network 201 is used for the image processing (step 104). Convolutional neural networks were originally designed to classify between different images. Their architecture follows specific rules and orders of layers, beginning with an input layer, and proceeding to one or more convolutional layers, pooling layers, and dense layers, also known as fully connected layers, as is known to those of skill in the art.

Feed-forward neural network 202 is used for evaluation of the metadata. Feedforward neural network 202 maps the different metadata onto an output function corresponding to an evaluation of the subject's understanding.

Because the system 10 is designed to produce one score for each reproduction, the convolutional neural network 201 and feedforward neural network 202 are combined at a shared dense layer phase 203. This dense layer phase issues a combined output 204. In exemplary embodiments, the combined output 204 is a ranking of the subject's gestalt understanding of the original drawing on a linear scale, such as a scale of 1 to 4, or an equivalent textual ranking. For example, the output may be one of the words mediocre (lowest level); poor (next higher level); good (next higher level); or perfect (highest level).

In addition to the combined evaluation, the system may output individual vectors corresponding to each of the metadata that was measured, for separate analysis of each of those metadata categories. The individual vectors may be used by the practitioner in order to consider the subject's proficiency in specific measured categories. For example, a subject may draw a perfect reproduction, but may take a long time or use an inordinate number of pen lifts to do so. Optionally, the individual metadata vectors may also be plotted on a scale of normal values for those parameters. These individual metadata vectors may provide context for the overall score output by the system, and may enable the practitioner to identify specific areas of strength and weakness in the subject.

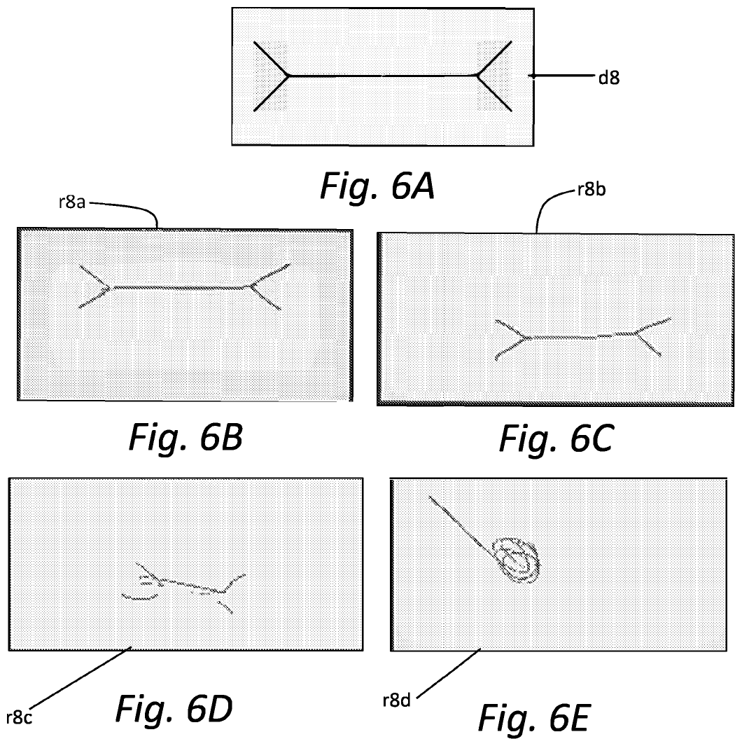

FIGS. 6A-6E depict examples of scoring of different reproductions that were performed by system 10. Original drawing d8 is shown in FIG. 6A, and four reproductions r8a, r8b, r8c, and r8d are shown in FIGS. 6B-6E. The system 10 assigned the reproduction r8a a value of “perfect,” assigned r8b a value of “good,” assigned r8c a value of “poor,” and assigned r8d a value of “mediocre.” The same images were also assigned to a human specialist for scoring. The human specialist reached the same initial conclusions for three of the reproductions, except that the specialist initially assigned reproduction r8c a value of “mediocre” rather than “weak.” After reviewing the results generated by the system, the specialist confirmed that the system indeed correctly classified the reproduction. Notably, the system 10 was able to identify not only the content of the reproduction (e.g., a line with two inverted arrows), which is a classic image processing task, but also rank the quality of the reproduction. Moreover, following training of the neural network, as will be described below, the system was able to perform this ranking even better than a trained human professional.

Returning to FIG. 4, at step 107, the method steps are repeated for a series of drawings. For example, as discussed above, the method may be repeated on a series of 20 drawings in which the subject is called upon to copy the drawing, as well as 20 drawings in which the subject is called upon to recall the drawing. Optionally, the scores for each drawing may be combined so as to arrive at a total score for the subject.

Prior to commencing method 100, it is necessary to train the neural network.

Typically, neural networks are trained with a supervised learning process. In a supervised learning model, the network is given a set of N training examples of the form {(X₁, y₁), . . . (Xₙ, Yₙ)}, wherein xᵢ is the feature vector of the i-th example and yᵢ is its label (i.e., class). A supervised learning algorithm seeks a function g:X→Y, wherein X is the input (feature) space and Y is the output (label) space. The feature space includes the drawn image itself, and the metadata parameters regarding the image, as discussed above.

For the supervised learning process to proceed, each supervised learning algorithm needs labeled samples. One exemplary manner to collect labeled samples is to assign a duplication score with a human specialist. For example, researchers may collect a data set including a plurality of sample reproductions of the original drawings. Two independent psychologists may rate each sample reproduction based on various parameters. This rating serves as the “true” label of each sample reproductions.

Following collection and labeling of the samples, the data is augmented. In machine learning, data augmentation is the process of synthetically modifying images without changing their essence. For example, a user may zoom, crop, skew, or rotate an image of a dog without changing it to an image of a cat. Classic data augmentation engages in these synthetic modifications while retaining the same true label of the drawing.

Augmentation is performed in order to increase the number of inputs for the neural network during a supervised learning process. Suppose, for example, that data gathering is conducted by administering 100 tests for multiple age levels of children. Because each test includes 40 drawings, the total number of drawings is 4,000 per age group. This quantity is usually vastly insufficient for a robust machine learning algorithm, let alone a convolutional neural network (CNN) with thousands of different parameters. Therefore, it is necessary to supplement the data with data augmentation.

A challenge of data augmentation in the context of the tests described in the present disclosure is that classic methods of data augmentation distort the images in a way that would change the scoring of those images. For example, image scaling (how big the drawing is relative to the original) and image rotation (how rotated the drawing is relative to the original) are two of the features that the neural network takes into account when computing an overall score. Therefore, one cannot modify these factors while maintaining the original score of the unscaled, unrotated image.

Accordingly, in order to utilize reliable data augmentation, two approaches may be used. First, it is possible to apply “gentle” modifications. Such gentle modifications may include one or more of scaling, pixel shifting, or horizontal flipping, in a manner that is sufficiently subtle so as not to affect a scoring of a given reproduction. Examples of such changes include 1-3% scaling, a 10 pixel shift to the image center, and horizontal image flips (for some images). These changes are hardly noticeable to the naked eye, but are considered different inputs from the perspective of the algorithm. A second approach is to modify the input images more drastically, and then to submit the modified images back to the human specialists for manual scoring.

Continuing to refer to FIG. 4, and specifically to step 108, a separate advantage of collecting metadata for each reproduction, beyond enhancing the understanding of each particular subject, is the ability to aggregate the data of multiple users. This aggregation may proceed separately from the process of scoring each particular user's reproductions. For example, the system 10 may aggregate the reaction times and performance times for a set of reproductions of a given original drawing. The result of the aggregation is a large data set of metadata corresponding to each original drawing.

At step 109, the system may determine a kernel density function for standard performance in one or more metadata categories. A kernel density function is a non-parametric way to estimate the probability density function of a random variable. As applied to the disclosed embodiments, the kernel density function may generate norms regarding the performance of one or more reproductions. The norms may include, for example, metrics regarding the mean and the standard deviation of metadata parameters, such as the average time it takes a child to duplicate a complex drawing. Such metrics do not exist for pen-and-paper tests, and, as a result, children's performance cannot be accurately evaluated as compared to their peers. The ability to generate such a kernel density function, for multiple parameters, represents a significant advance in its own right, made possible by implementation of the assessment test on a tablet.

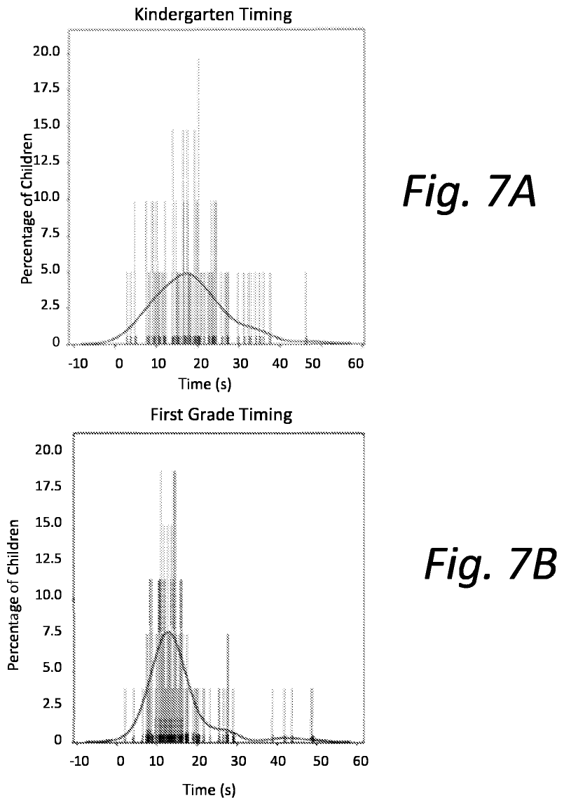

Exemplary kernel density functions are shown in FIG. 7A and FIG. 7B. FIG. 7A illustrates a kernel density estimation of the time spent for kindergarten children to reproduce a particular drawing. The x-axis is the time, in seconds, and the y-axis is the estimation of a percentage of children to complete the reproduction in a given time. FIG. 7B illustrates the kernel density estimation of the time spent for reproduction of the same drawing, by first-grade children. As can be seen, and as expected, the peak of the kindergarten data is a few seconds after the peak of the first grade data. From the kernel density estimation, it is possible to derive norms for expected time of completion for a given age.

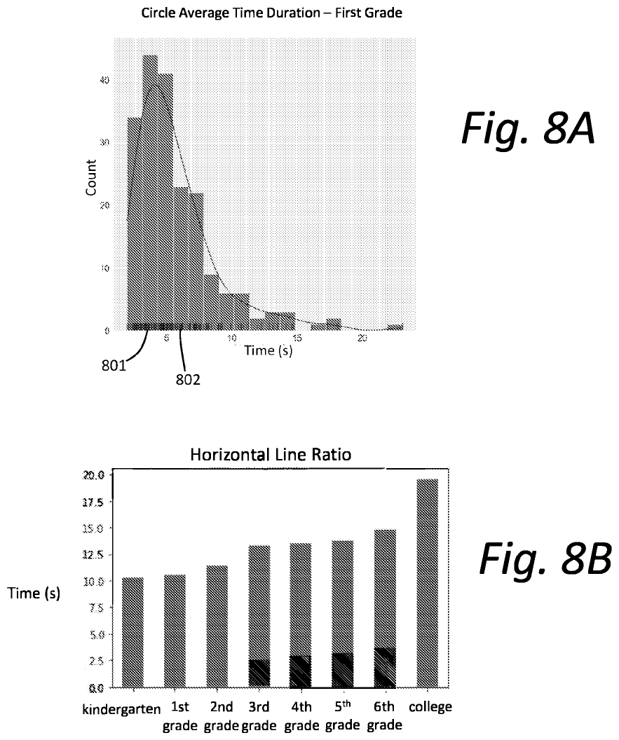

The software produces a norm graph for each feature of each shape. For example, in FIG. 8A, one can see an output graph of average time duration of a specific child relative to the norm established for that child's peer group. The child's time of approximately 2 seconds is indicated at point 801, while the mean time for children of that age group, which is over 5 seconds, is indicated at point 802.

FIG. 8B illustrates the generation of different norms for the same task among different ages, and in particular shows the improvement in shape accuracy as the student gets older. The metric illustrated is horizontal line ratio. The shape accuracy gradually improves with each age group, as expected, whereby the highest gap is between sixth graders and college students. This graph thus validates the ability of the data to generate norms-different age groups do possess different norms and those norms can be accurately measured.

1-26. (canceled)

27. A method of evaluating a subject's visuospatial ability, comprising:
displaying an original drawing on a screen;
receiving input on the screen from a stylus, corresponding to a reproduction of the drawing by the subject;
during the receiving step, collecting metadata regarding the process of the reproduction;
comparing a resemblance of the reproduction to the original drawing;
evaluating the reproduction and the metadata to infer therefrom the subject's understanding of the drawing; and
issuing a combined evaluation of the subject's gestalt understanding of the drawing based on a combination of the resemblance comparison and the metadata evaluation.

28. The method of claim 27, wherein the metadata include a reaction time from displaying of the original drawing until commencement of reproduction of the drawing, a performance time from commencement of reproduction until completion of reproduction.

29. The method of claim 27, wherein the metadata include a number of stylus strokes used to reproduce the drawing.

30. The method of claim 27, wherein the metadata include pressure exerted by the subject with the stylus onto the screen during reproduction.

31. The method of claim 27, wherein the metadata include azimuth angle of the stylus on the screen during reproduction.

32. The method of claim 27, wherein the metadata include age of the subject.

33. The method of claim 27, wherein the comparison of resemblance is based on at least: position of reproduction on the screen, scale of reproduction, orientation of reproduction, and number of elements in the reproduction.

34. The method of claim 27, further comprising repeating each step with a series of predefined original drawings.

35. The method of claim 34, wherein the metadata include cumulative time of completion of all the predefined original drawings in the series.

36. The method of claim 27, further comprising: performing the method with a plurality of unique subjects; aggregating the metadata collected from each subject; and, based on the aggregated metadata, determining a kernel density function for performance in one or more measured metadata categories, and deriving a norm for standard performance from the kernel density function.

37. The method of claim 27, further comprising: comparing the resemblance with a convolutional neural network; evaluating the metadata with a feed-forward neural network; and combining the convolutional neural network and the feed forward neural network at a dense layer phase.

38. The method of claim 37, further comprising training the convolutional neural network, the training step comprising: collecting a data set comprising a plurality of sample reproductions of the original drawings; and manually assigning a similarity score to each reproduction.

39. The method of claim 38, further comprising augmenting the data set with modified sample reproductions, said augmenting comprising at least one of changing scaling, shifting pixels, and horizontal flipping, in a manner that is sufficiently subtle not to affect a scoring of a given reproduction.

40. The method of claim 27, wherein the combined evaluation is a ranking of the subject's gestalt understanding of the original drawing.

41. A system for evaluating a subject's visuospatial ability, comprising:
a mobile computing device including a touch screen configured to display images and to receive input from a stylus; a processor, and a non-transitory computer readable medium;
and a computer program product embodied on the non-transitory computer readable medium, that, when executed by the processor, causes the processor to perform the following steps:
displaying an original drawing on the screen;
during receipt of input on the screen from the stylus, corresponding to a reproduction of the drawing by the subject; collecting metadata regarding the process of the reproduction;
comparing a resemblance of the reproduction to the original drawing; evaluating the reproduction and the metadata to infer therefrom the subject's understanding of the drawing; and
issuing a combined evaluation of the subject's gestalt understanding of the drawing based on a combination of the resemblance comparison and the metadata evaluation.

42. The system of claim 41, wherein the metadata include a reaction time from displaying of the original drawing until commencement of reproduction of the drawing, and a performance time from commencement of reproduction until completion of reproduction.

43. The system of claim 41, wherein the metadata include a number of stylus strokes used to reproduce the drawing.

44. The system of claim 41, wherein the metadata include pressure exerted by the subject with the stylus onto the screen during reproduction.

45. The system of claim 41, wherein the metadata include azimuth angle of the stylus on the screen during reproduction.

46. The system of claim 41, wherein the computer program product is configured to compare the resemblance based on at least: position of reproduction on the screen, scale of reproduction, orientation of reproduction, and number of elements in the reproduction.
